(19) United States
(12) Patent Application Publication
(10) Pub. No.: US 2025/0197592 A1
(43) Pub. Date: Jun. 19, 2025

(54) PROCESS FOR HYDROLYTICALLY DEPOLYMERIZING A POLYAMIDE

(71) Applicant: BASF SE, Ludwigshafen am Rhein (DE)

(72) Inventors: Stefan BLEI, Ludwigshafen am Rhein (DE); Faissal-Ali EL-TOUFAILI, Ludwigshafen am Rhein (DE); Oliver BEY, Ludwigshafen am Rhein (DE); Vikram Raghavendhar RAVIKUMAR, Ludwigshafen am Rhein (DE); Bart VANDER STRAETEN, Antwerpen (BE); Michael SCHREIBER, Ludwigshafen am Rhein (DE)

(21) Appl. No.: 18/851,246

(22) PCT Filed: Mar. 30, 2023
(86) PCT No.: PCT/EP2023/058274

§ 371 Date: Sep. 26, 2024

(30) Foreign Application Priority Data
Mar. 30, 2022 (EP) 22165486.6

(51) Int. Cl. C08J 11/14; B01J 8/00; B01J 8/08; C07D 223/10

(52) U.S. Cl. CPC C08J 11/14; B01J 8/003; B01J 8/085; C07D 223/10; C08J 2377/02

(57) ABSTRACT

The present invention relates to a process for hydrolytically depolymerizing a polyamide prepared from ε-caprolactam and an apparatus for carrying out a process for hydrolytically depolymerizing a polyamide prepared from ε-caprolactam.

The present invention relates to a process for hydrolytically depolymerizing a polyamide prepared from ε-caprolactam and an apparatus for carrying out a process for hydrolytically depolymerizing a polyamide prepared from ε-caprolactam, preferably for carrying out the aforementioned process.

Polyamide, and in particular polyamide 6 being characterized by the formula (—NH—(CH₂)₅—CO—)ₙ, can be found in numerous materials, such as packaging, engineering plastics from automotive and textile filaments. The latter represents about 40% of the polyamide 6 global market. At present, only a very small part of the textile filaments is recycled while it represents a significant percentage of the global CO₂ emissions. There is thus a need to recycle polyamide 6 from such materials. Processes for alkaline depolymerizing a polyamide exists. Thus, there is a need to provide an improved process for depolymerizing a polyamide able to overcome these issues.

Surprisingly, it was found that the process of the present invention according to which polyamide is hydrolytically hydrolyzed is more robust and is cheaper. Using a hydrolytic process compared to an alkaline depolymerization process permits to reduce the CO₂ footprint.

Therefore, the present invention relates to a process for hydrolytically depolymerizing a polyamide prepared from ε-caprolactam, said polyamide being contained in a solid material M, the process comprising
(i) providing the solid material M containing the polyamide, M having a temperature T_{M}, wherein T_{M}<T_{P}, T_{P} being the melting point of the polyamide;
(ii) providing a liquid aqueous stream S_{W}, wherein from 90 to 100 weight-% of S_{W} consist of water and wherein S_{W} has a temperature T_{SW}, wherein T_{SW}>T_{P};
(iii) feeding the solid material M provided according to (i) and the liquid aqueous stream S_{W} provided according to (ii) into a chemical reactor unit R_{U}, obtaining a mixture;
(iv) subjecting the mixture obtained according to (iii) in R_{U} to a depolymerization temperature T_{D} at a depolymerization pressure p_{D}, wherein T_{M}<T_{D}<T_{SW}, obtaining an aqueous mixture containing ε-caprolactam dissolved in water;
(v) removing an aqueous liquid stream S_{R} from R_{U}, S_{R} containing ε-caprolactam dissolved in water.

Preferably, T_{D} is in the range of from 230 to 320° C., more preferably in the range of from 250 to 300° C., more preferably in the range of from 270 to 295° C.

Preferably, T_{SW} is in the range of from 240 to 350° C., more preferably in the range of from 260 to 330° C., more preferably in the range of from 290 to 325° C.

The excess heat of the liquid aqueous stream S_{W} (T_{SW}) melts the solid material M containing the polyamide which is preferably in the form of granules, and provides the needed reaction enthalpy.

Any additional heat required to maintain the reaction temperature can be provided through a heating jacket of the reactor unit R_{U}, using hot oil as heating medium.

Preferably, ΔT=T_{SW}−T_{D} is in the range of from 10 to 70° C., more preferably in the range of from 10 to 50° C., more preferably in the range of from 10 to 30° C.

Preferably, p_{D} is in the range of from 40 to 120 bar, more preferably in the range of from 50 to 100 bar, more preferably in the range of from 60 to 90 bar.

Preferably from 91 to 100 weight-%, more preferably from 92 to 100 weight-%, more preferably from 95 to 100 weight-%, of S_{W} consist of water.

Preferably, the solid material M comprises, more preferably consists of, waste material, wherein said waste material more preferably comprises textile waste material.

Preferably from 10 to 99 weight-%, more preferably from 30 to 98.5 weight-%, more preferably from 50 to 98 weight-%, more preferably from 80 to 98 weight-%, of M consist of the polyamide; or preferably from 10 to 100 weight-%, more preferably from 30 to 100 weight-%, more preferably from 50 to 100 weight-%, more preferably from 80 to 100 weight-%, of M consist of the polyamide.

Preferably, M is in the form of granules, wherein the mean diameter of the granules is preferably in the range of from 0.5 to 10 mm, more preferably in the range of from 1 to 7 mm, more preferably in the range of from 2 to 4 mm.

According to (iii), M and S_{W} are preferably fed into R_{U} at a mixing ratio m_{W}/kg:m_{P}/kg, defined as the amount of water contained in S₁, m_{W}, relative to the mass of polyamide contained in M, m_{P}, in the range of from 1:1 to 20:1, more preferably in the range of from 2:1 to 15:1, more preferably in the range of from 5:1 to 10:1.

The mixing is preferably ensured by dosing the amount of water (S_{W}) stepwise with ongoing reaction time, and dosing the solid material M in the reactor unit R_{U}.

Preferably, M and S_{W} are fed into R_{U} one after the other. More preferably, S_{W} is fed into R_{U} and subsequently M is fed into R_{U} containing S_{W}.

Prior to being fed into R_{U}, M is neither subjected to melting nor subjected to mashing.

Preferably, the chemical reactor unit R_{U} comprises z chemical reactors Rᵢ, i=1 . . . z, wherein z is in the range of from 1 to 10, more preferably in the range of from 2 to 8, more preferably in the range of from 2 to 6, more preferably in the range of from 2 to 5, more preferably in the range of from 2 to 4, more preferably 3 or 4, more preferably 4.

Preferably, z>1 and at least two reactors Rᵢ, more preferably the z reactors Rᵢ, are serially coupled.

Preferably, the z reactors Rᵢ are serially coupled, wherein
according to (iii), M and S_{W} are fed into Rᵢ, i=1;
an aqueous liquid stream Sᵢ containing ε-caprolactam dissolved in water is removed from Rᵢ and fed into Rᵢ₊₁, i<Z;
according to (v), S_{R} is removed from R_{z};
   wherein in every reactor Rᵢ, a depolymerization temperature T_{Di} at a depolymerization pressure p_{Di} are maintained, wherein, independently of each other, T_{Di} is in the range of from 230 to 320° C., more preferably in the range of from 250 to 300° C., more preferably in the range of from 270 to 295° C., and wherein, independently of each other, p_{Di} is preferably in the range of from 40 to 120 bar, more preferably in the range of from 50 to 100 bar, more preferably in the range of from 60 to 90 bar.

Preferably, maintaining a depolymerization temperature T_{Di} in a reactor Rᵢ comprises heating the reactor contents of Rᵢ, more preferably indirectly heating the reactor contents of Rᵢ, wherein more preferably, maintaining a depolymerization temperature T_{Di} in a reactor Rᵢ comprises heating the reactor contents of Rᵢ by passing a heating medium through a heating jacket of Rᵢ. The heating medium is preferably hot oil. However, it is noted that other heating media known by the skilled person can be used for passing through the heating jacket of Rᵢ.

Preferably, the z reactors Rᵢ are vertically arranged, with R₁ being the top-most reactor and R_{z} being the bottom-most reactor, wherein Sᵢ obtained from Rᵢ is transferred to Rᵢ₊₁ by gravity, more preferably by gravity only.

Preferably, at least 1, more preferably z reactors Rᵢ, are configured as non-stirred reactors or as non-circulation reactors, more preferably as non-stirred reactors and non-circulation reactors.

The system is preferably operated without stirrer and without circulation pump. That avoids difficult sealings, i.e. high pressure sealings, for the stirrer and pump.

As mentioned in the foregoing, the mixing is preferably ensured by dosing the amount of water stepwise with ongoing reaction time, and dosing the liquid solution, for example, from reactor R₁ by gravity to reactor R₂ and R₃ after parts of the reaction time.

Preferably, the reactors R₁ to R_{y-1} are operated in batch mode and the reactors R_{y} to R_{z} are operated in continuous mode, wherein y>1 and y≤z, wherein y is more preferably z.

Preferably, the residence time in one or more of the reactors R₁ to R_{y-1}, more preferably in the reactors R₁ to R_{y-1}, is in the range of from 5 to 40 minutes, more preferably in the range of from 10 to 30 minutes, more preferably in the range of from 15 to 25 minutes.

Preferably, the residence time in the reactors R_{y} to R_{z}, wherein y is more preferably z, is in the range of from 1 second to 40 minutes, more preferably in the range of from 2 seconds to 30 minutes, more preferably in the range of from 3 seconds to 25 minutes.

Preferably, the overall residence time in the chemical reactor unit is in the range of from 15 to 160 minutes, more preferably in the range of from 30 to 120 minutes, more preferably in the range of from 45 to 100 minutes, more preferably in the range of from 60 to 80 minutes.

More preferably, four identical chemical reactors R₁-R₄ are arranged in series. The first three, R₁-R₃, are operated in batch mode, all of said reactors having low residence time and the last, R₄, in continuous mode to enable continuous feeding of the downstream process steps.

As to the feeding of S_{W} and M, it is preferred that M and S_{W} are fed into Rᵢ one after the other. More preferably, S_{W} is fed into Rᵢ and subsequently M is fed into Rᵢ containing S_{W}. After a time T, Sᵢ is removed from Rᵢ and fed to Rᵢ₊₁.

Preferably, providing a liquid aqueous stream S_{W} according to (ii) comprises
(ii.1) separating at least a part of the water contained in the aqueous liquid stream S_{R} removed from R_{U};
(ii.2) feeding at least a part of the water separated according to (ii.1) back into the process as part of the stream S_{W}.

This is in particular realized during the on-going process, this permits to recycle water which permits to reduce environmental footprint and reducing costs.

Preferably, providing the solid material M containing the polyamide according to (i) comprises
(i.1) providing the solid material in a first zone of a two-zone receiving and discharge means RD_{M}, wherein the temperature of M in said first zone is in the range of from −10 to 50° C., more preferably in the range of from 15 to 40° C., more preferably in the range of from 20 to 30° C., more preferably at atmospheric pressure;
(i.2) passing the solid material provided in the first zone to a second zone of the RD_{M}, wherein said second zone comprises outlet means being connected via a connecting line with the reactor unit R_{U};
(i.3) feeding a gas stream into the second zone of the RD_{M} comprising the solid material, said gas stream having a pressure p_{G} with p_{G}>p_{D}, wherein p_{G} is more preferably in the range of p_{D}<P_{G}≤(p_{D}+5 bar), wherein the gas stream has a temperature more preferably in the range of from 10 to 40° C., more preferably in the range of from 15 to 30° C.;
(i.4) passing the solid material from the second zone of the RD_{M} into the connecting line according to (i.2);
   wherein the two-zone receiving and discharge means RD_{M} is more preferably a two-zone hopper. The raw material or feed, namely the solid material M, is preferably delivered as granules into silos using trucks and/or big bags. The silos could be under N₂ atmosphere to prevent the risk of dust explosion. Granules are preferably fed pneumatically from these silos to a two-zone receiving and discharge means RD_{M}, preferably a hopper, more preferably a two-zone hopper. It is also conceivable that a big bag unloading station discharges directly into the hopper.

Preferably, the receiving and discharge means RD_{M}, more preferably a two-zone hopper, unloads the feed mixture, namely the solid material M, into the reactor unit R_{U}, more preferably into the first reactor R₁ of the reactor unit, using gravity.

Preferably, the gas stream according to (i.3) comprises one or more of nitrogen and water,

The present invention further relates to an apparatus for carrying out a process for hydrolytically depolymerizing a polyamide prepared from ε-caprolactam, said polyamide being contained in a solid material M, preferably for carrying out a process according to the present invention, the apparatus comprising
(a) a reactor unit R_{U} comprising z chemical reactors Rᵢ, i=1 . . . z, wherein z is in the range of from 2 to 8, more preferably in the range of from 2 to 6, more preferably in the range of from 2 to 5, more preferably in the range of from 2 to 4, more preferably 3 or 4, more preferably 4;
   wherein the z reactors Rᵢ are serially coupled and vertically arranged, with R₁ being the top-most reactor and R_{z} being the bottom-most reactor;
   wherein R₁ comprises inlet means I_{M} for feeding the solid material M into R₁ and inlet means I_{SW} for feeding a liquid aqueous stream S_{W} into R₁, said inlet means being located at the top of R₁;
   wherein every reactor Rᵢ, comprises outlet means O_{Ri} for removing an aqueous liquid product stream Sᵢ from Rᵢ;
   wherein a reactor Rᵢ, i>1, comprises inlet means I_{Ri} for feeding the aqueous liquid product stream Sᵢ₋₁ removed from Rᵢ₋₁ via a connecting line Cᵢ₋₁ into Rᵢ;
   wherein the outlet stream removed from R_{z} is an aqueous liquid stream S_{R}, S_{R} containing ε-caprolactam dissolved in water.

Preferably at least one reactor Rᵢ, more preferably all reactors Rᵢ, are equipped with a heating jacket for passing a heating medium through said jacket. The heating medium is preferably hot oil. It is noted that other heating medium known the skilled person can be used for being passed through the heating jacket of Rᵢ.

Preferably at least 1, more preferably z reactors Rᵢ, are configured as non-stirred reactors or as non-circulation reactors, more preferably as non-stirred reactors and non-circulation reactors.

Preferably at least one connecting line Cᵢ, more preferably all z−1 connecting lines Cᵢ are configured to allow transferring Sᵢ₋₁ from Rᵢ₋₁ to Rᵢ by gravity, more preferably by gravity only.

Preferably at least one connecting line Cᵢ, more preferably all z−1 connecting lines Cᵢ are not equipped with pumping means, more preferably not equipped with conveyor means.

Preferably the reactors R₁ to R_{y-1} are batch mode reactors and the reactors R_{y} to R_{z} are continuous mode reactors, wherein y<1 and y≤z, wherein y is more preferably z.

Preferably the apparatus further comprises
(b) separating means SE_{W} for separating water from S_{R};
   wherein said separating means SE_{W} is arranged downstream of R_{U}, comprises inlet means I_{SE} for passing S_{R} into SE_{W}, the I_{SE} being connected via a connecting line with the outlet means O_{R}, of the reactor R_{z}; and further comprise outlet means O_{SE} for removing water from SE_{W}, the O_{SE} being connected via a connecting line with the inlet means I_{SW} of the reactor R₁.

Preferably the apparatus further comprises
(c) a two-zone receiving and discharge means RD_{M} for metering the solid material M to the reactor unit R_{U};
   wherein said two-zone receiving and discharge means RD_{M} is arranged upstream of R_{U}, wherein the first zone of RD_{M} is arranged vertically above the second zone of RD_{M} and comprises inlet means I_{RD} for receiving the solid material M, wherein the second zone of RD_{M} comprises outlet means O_{RD} for removing the solid material from RD_{M}, the outlet means O_{RD} being connected via a connecting line with the inlet means I_{M} of the reactor R₁, wherein the RD_{M} further comprises closing and opening means arranged between the first zone and the second zone of the RD_{M}.

Preferably the second zone comprises inlet means I_{G} for feeding a gas stream, more preferably a high-pressure gas stream, into the second zone.

Preferably the apparatus is arranged vertically above the reactor unit R_{U}, wherein the connecting line between O_{RD} and I_{M} is configured to allow transferring M from RD_{M} to R₁ by gravity, wherein said connecting line is optionally equipped with a conveyor means, more preferably a feeder means, more preferably a rotary feeder means. More preferably, RD_{M} is a hopper.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.
1. A process for hydrolytically depolymerizing a polyamide prepared from ε-caprolactam, said polyamide being contained in a solid material M, the process comprising
   (i) providing the solid material M containing the polyamide, M having a temperature T_{M}, wherein T_{M}<T_{P}, T_{P} being the melting point of the polyamide;
   (ii) providing a liquid aqueous stream S_{W}, wherein from 90 to 100 weight-% of S_{W} consist of water and wherein S_{W} has a temperature T_{SW}, wherein T_{SW}>T_{P};
   (iii) feeding the solid material M provided according to (i) and the liquid aqueous stream S_{W} provided according to (ii) into a chemical reactor unit R_{U}, obtaining a mixture;
   (iv) subjecting the mixture obtained according to (iii) in R_{U} to a depolymerization temperature T_{D} at a depolymerization pressure p_{D}, wherein T_{M}<T_{D}<T_{SW}, obtaining an aqueous mixture containing ε-caprolactam dissolved in water;
   (v) removing an aqueous liquid stream S_{R} from R_{U}, S_{R} containing ε-caprolactam dissolved in water.
2. The process of embodiment 1, wherein T_{D} is in the range of from 230 to 320° C., preferably in the range of from 250 to 300° C., more preferably in the range of from 270 to 295° C.
3. The process of embodiment 1 or 2, wherein T_{SW} is in the range of from 240 to 350° C., preferably in the range of from 260 to 330° C., more preferably in the range of from 290 to 325° C.
4. The process of any one of embodiments 1 to 3, wherein ΔT=T_{SW}−T_{D} and ΔT is in the range of from 10 to 70° C., preferably in the range of from 10 to 50° C., more preferably in the range of from 10 to 30° C.
5. The process of any one of embodiments 1 to 4, wherein p_{D} is in the range of from 40 to 120 bar, preferably in the range of from 50 to 100 bar, more preferably in the range of from 60 to 90 bar.
6. The process of any one of embodiments 1 to 5, wherein from 91 to 100 weight-%, preferably from 92 to 100 weight-%, more preferably from 95 to 100 weight-%, of S_{W} consist of water.
7. The process of any one of embodiments 1 to 6, wherein M comprises, preferably consists of waste material, wherein said waste material preferably comprises textile waste material.
8. The process of any one of embodiments 1 to 7, wherein from 10 to 99 weight-%, more preferably from 30 to 98.5 weight-%, more preferably from 50 to 98 weight-%, more preferably from 80 to 98 weight-%, of M consist of the polyamide.
9. The process of any one of embodiments 1 to 8, wherein M is in the form of granules, wherein the mean diameter of the granules is preferably in the range of from 0.5 to 10 mm, more preferably in the range of from 1 to 7 mm, more preferably in the range of from 2 to 4 mm.
10. The process of any one of embodiments 1 to 9, wherein according to (iii), M and S_{W} are fed into R_{U} at a mixing ratio m_{W}/kg:m_{P}/kg, defined as the amount of water contained in S₁, m_{W}, relative the mass of polyamide contained in M, m_{P}, in the range of from 1:1 to 20:1, preferably in the range of from 2:1 to 15:1, more preferably in the range of from 5:1 to 10:1.
11. The process of any one of embodiments 1 to 10, wherein M and S_{W} are fed into R_{U} one after the other, wherein, preferably, S_{W} is fed into R_{U} and subsequently M is fed into R_{U} containing S_{W}.
12. The process of any one of embodiments 1 to 11, wherein prior to being fed into R_{U}, M is neither subjected to melting nor subjected to mashing.
13. The process of any one of embodiments 1 to 12, wherein the chemical reactor unit R_{U} comprises z chemical reactors Rᵢ, i=1 . . . z, wherein z is in the range of from 1 to 10, preferably in the range of from 2 to 8, more preferably in the range of from 2 to 6, more preferably in the range of from 2 to 5, more preferably in the range of from 2 to 4, more preferably 3 or 4, more preferably 4.
14. The process of embodiment 13, wherein z>1 and wherein at least 2 reactors Rᵢ, preferably z reactors Rᵢ are serially coupled.
15. The process of embodiment 14, wherein z reactors Rᵢ are serially coupled, wherein
   according to (iii), M and S_{W} are fed into Rᵢ, i=1;
   an aqueous liquid stream Sᵢ containing ε-caprolactam dissolved in water is removed from Rᵢ and fed into Rᵢ₊₁, i<z;
   according to (v), S_{R} is removed from R_{z};
      wherein in every reactor Rᵢ, a depolymerization temperature T_{Di} at a depolymerization pressure p_{Di} is maintained, wherein, independently of each other, T_{Di} is in the range of from 230 to 320° C., preferably in the range of from 250 to 300° C., more preferably in the range of from 270 to 295° C., and wherein, independently of each other, p_{Di} is preferably in the range of from 40 to 120 bar, more preferably in the range of from 50 to 100 bar, more preferably in the range of from 60 to 90 bar.
16. The process of embodiment 15, wherein maintaining a depolymerization temperature T_{Di} in a reactor Rᵢ comprises heating the reactor contents of Rᵢ, preferably indirectly heating the reactor contents of Rᵢ, wherein more preferably, maintaining a depolymerization temperature T_{Di} in a reactor Rᵢ comprises heating the reactor contents of Rᵢ by passing a heating medium through a heating jacket of Rᵢ.
17. The process of embodiment 15 or 16, wherein the z reactors Rᵢ are vertically arranged, with R₁ being the top-most reactor and R_{z} being the bottom-most reactor, wherein Sᵢ obtained from Rᵢ is transferred to Rᵢ₊₁ by gravity, preferably by gravity only.
18. The process of any one of embodiments 13 to 17, preferably of embodiment 18, wherein at least 1, preferably z reactors Rᵢ are configured as non-stirred reactors or as non-circulation reactors, preferably as non-stirred reactors and non-circulation reactors.
19. The process of any one of embodiments 13 to 18, wherein the reactors R₁ to R_{y-1} are operated in batch mode and the reactors R_{y} to R_{z} are operated in continuous mode, wherein y>1 and y≤z, wherein y is preferably z.
20. The process of embodiment 19, wherein the residence time in one or more of the reactors R₁ to R_{y-1}, preferably in the reactors R₁ to R_{y-1}, is in the range of from 5 to 40 minutes, more preferably in the range of from 10 to 30 minutes, more preferably in the range of from 15 to 25 minutes; wherein the residence time in the reactor R_{y}, wherein y is preferably z, is preferably in the range of from 1 second to 40 minutes, more preferably in the range of from 2 seconds to 30 minutes, more preferably in the range of from 3 seconds to 25 minutes.
21. The process of embodiment 19 or 20, wherein the overall residence time in the chemical reactor unit is in the range of from 15 to 160 minutes, preferably in the range of from 30 to 120 minutes, more preferably in the range of from 45 to 100 minutes, more preferably in the range of from 60 to 80 minutes.
22. The process of any one of embodiments 1 to 21, wherein providing a liquid aqueous stream S_{W} according to (ii) comprises
   (ii.1) separating at least a part of the water contained in the aqueous liquid stream S_{R} removed from R_{U};
   (ii.2) feeding at least a part of the water separated according to (ii.1) back into the process as part of the stream S_{W}.
23. The process of any one of embodiments 1 to 22, wherein providing the solid material M containing the polyamide according to (i) comprises
   (i.1) providing the solid material in a first zone of a two-zone receiving and discharge means RD_{M}, wherein the temperature of M in said first zone is in the range of from −10 to 50° C., preferably in the range of from 15 to 40° C., more preferably in the range of from 20 to 30° C., preferably at atmospheric pressure;
   (i.2) passing the solid material provided in the first zone to a second zone of the RD_{M}, wherein said second zone comprises outlet means being connected via a connecting line with the reactor unit R_{U};
   (i.3) feeding a gas stream into the second zone of the RD_{M} comprising the solid material, said gas stream having a pressure p_{G} with p_{G}>p_{D}, wherein p_{G} is preferably in the range of p_{D}<p_{G}≤(p_{D}+5 bar), wherein the gas stream has a temperature preferably in the range of from 10 to 40° C., more preferably in the range of from 15 to 30° C.;
   (i.4) passing the solid material from the second zone of the RD_{M} into the connecting line according to (i.2).
24. The process of embodiment 23, wherein the two-zone receiving and discharge means RD_{M} is a two-zone hopper.
25. The process of embodiment 23 or 24, wherein the gas stream according to (i.3) comprises one or more of nitrogen and water.
26. An apparatus for carrying out a process for hydrolytically depolymerizing a polyamide prepared from ε-caprolactam, said polyamide being contained in a solid material M, preferably for carrying out a process according to any one of embodiments 1 to 25, the apparatus comprising
   (a) a reactor unit R_{U} comprising z chemical reactors Rᵢ, i=1 . . . z, wherein z is in the range of from 2 to 8, preferably in the range of from 2 to 6, more preferably in the range of from 2 to 5, more preferably in the range of from 2 to 4, more preferably 3 or 4, more preferably 4;
      wherein the z reactors Rᵢ are serially coupled and vertically arranged, with R₁ being the top-most reactor and R_{z} being the bottom-most reactor;
      wherein R₁ comprises inlet means I_{M} for feeding the solid material M into R₁ and inlet means I_{SW} for feeding a liquid aqueous stream S_{W} into R₁, said inlet means being located at the top of R₁;
      wherein every reactor Rᵢ, comprises outlet means O_{Ri} for removing an aqueous liquid product stream Sᵢ from Rᵢ;
      wherein a reactor Rᵢ, i>1, comprises inlet means I_{Ri} for feeding the aqueous liquid product stream Sᵢ₋₁ removed from Rᵢ₋₁ via a connecting line Cᵢ₋₁ into Rᵢ;
      wherein the outlet stream removed from R_{z} is an aqueous liquid stream S_{R}, S_{R} containing ε-caprolactam dissolved in water.
27. The apparatus of embodiment 26, wherein at least one reactor Rᵢ, preferably all reactors Rᵢ are equipped with a heating jacket for passing a heating medium through said jacket.
28. The apparatus of embodiment 26 or 27, wherein at least 1, preferably z reactors Rᵢ are configured as non-stirred reactors or as non-circulation reactors, preferably as non-stirred reactors and non-circulation reactors.
29. The apparatus of any one of embodiments 26 to 28, wherein at least one connecting line Cᵢ, preferably all z−1 connecting lines Cᵢ are configured to allow transferring Sᵢ₋₁ from Rᵢ₋₁ to Rᵢ by gravity, preferably by gravity only.
30. The apparatus of embodiment 29, wherein at least one connecting line Cᵢ, preferably all z−1 connecting lines Cᵢ are not equipped with pumping means, preferably not equipped with conveyor means.
31. The apparatus of any one of embodiments 26 to 30, wherein the reactors R₁ to R_{y-1} are batch mode reactors and the reactors R_{y} to R_{z} are continuous mode reactors, wherein y<1 and y≤z, wherein y is preferably z.
32. The apparatus of any one of embodiments 26 to 31, further comprising
   (b) separating means SE_{W} for separating water from S_{R};
      wherein said separating means SE_{W} is arranged downstream of R_{U}, comprises inlet means I_{SE} for passing S_{R} into SE_{W}, the I_{SE} being connected via a connecting line with the outlet means O_{R}, of the reactor R_{z}; and further comprise outlet means O_{SE} for removing water from SE_{W}, the O_{SE} being connected via a connecting line with the inlet means I_{SW} of the reactor R₁.
33. The apparatus of any one of embodiments 26 to 32, further comprising
   (c) a two-zone receiving and discharge means RD_{M} for metering the solid material M to the reactor unit R_{U};
      wherein said two-zone receiving and discharge means RD_{M} is arranged upstream of R_{U}, wherein the first zone of RD_{M} is arranged vertically above the second zone of RD_{M} and comprises inlet means I_{RD} for receiving the solid material M, wherein the second zone of RD_{M} comprises outlet means O_{RD} for removing the solid material from RD_{M}, the outlet means O_{RD} being connected via a connecting line with the inlet means I_{M} of the reactor R₁, wherein the RD_{M} further comprises closing and opening means arranged between the first zone and the second zone of the RD_{M}.
34. The apparatus of embodiment 33, wherein the second zone comprises inlet means I_{G} for feeding a gas stream, preferably a high-pressure gas stream, into the second zone.
35. The apparatus of embodiment 33 or 34, being arranged vertically above the reactor unit R_{U}, wherein the connecting line between O_{RD} and I_{M} is configured to allow transferring M from RD_{M} to R₁ by gravity, wherein said connecting line is optionally equipped with a conveyor means, preferably a feeder means, more preferably a rotary feeder means.
36. The apparatus of embodiment 35, wherein the RD_{M} is a hopper.

In the context of the present invention, it is noted that the term “polyamide prepared from ε-caprolactam” as used herein refers to “polyamide 6” being characterized by the formula (—NH—(CH₂)₅—CO—)ₙ. The term “bar” as used in the context of the present invention refers to “bar(abs)”, i.e. bar (absolute), sometimes also referred to “bara”.

The term “textile material” covers textile raw materials and non-textile raw materials that are processed by various methods into linear, planar and spatial structures. It concerns the linear textile structures produced from them, such as yarns, twisted yarns and ropes, the sheet-like textile structures, such as woven fabrics, knitted fabrics, braids, stitch-bonded fabrics, nonwovens and felts, and the three-dimensional textile structures, i.e. body structures, such as textile hoses, stockings or textile semi-finished products; and it further concerns those finished products which, using the aforementioned products, are brought into a saleable condition by making up, opening up and/or other operations for onward transmission to the processor, the trade or the end consumer.

The term “textile waste material” covers a textile material as defined above, the inherent value of which has been consumed from the perspective of its current holder and, thus, is an end-of-life material for said holder.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transfer to above abstract term to a concrete example, e.g. where X is a chemical element and A, B and C are concrete elements such as Li, Na, and K, or X is a temperature and A, B and C are concrete temperatures such as 10° C., 20° C., and 30° C. In this regard, it is further noted that the skilled person is capable of extending the above term to less specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and D, or B and C, or B and D, or C and D, or A and B and C, or A and B and D, or B and C and D, or A and B and C and D.

### DESCRIPTION OF THE FIGURES

FIG. 1 is a schematic representation of an apparatus used for the process according to the invention.

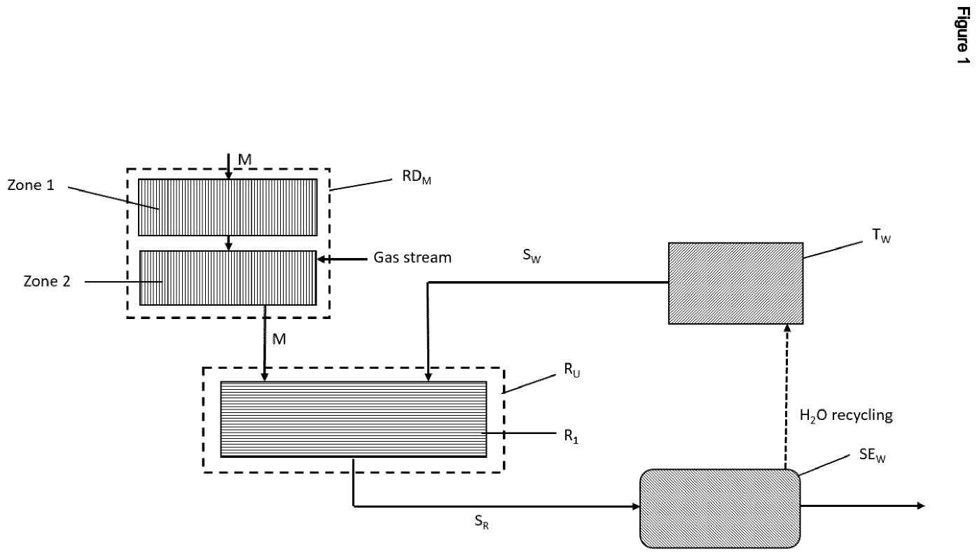

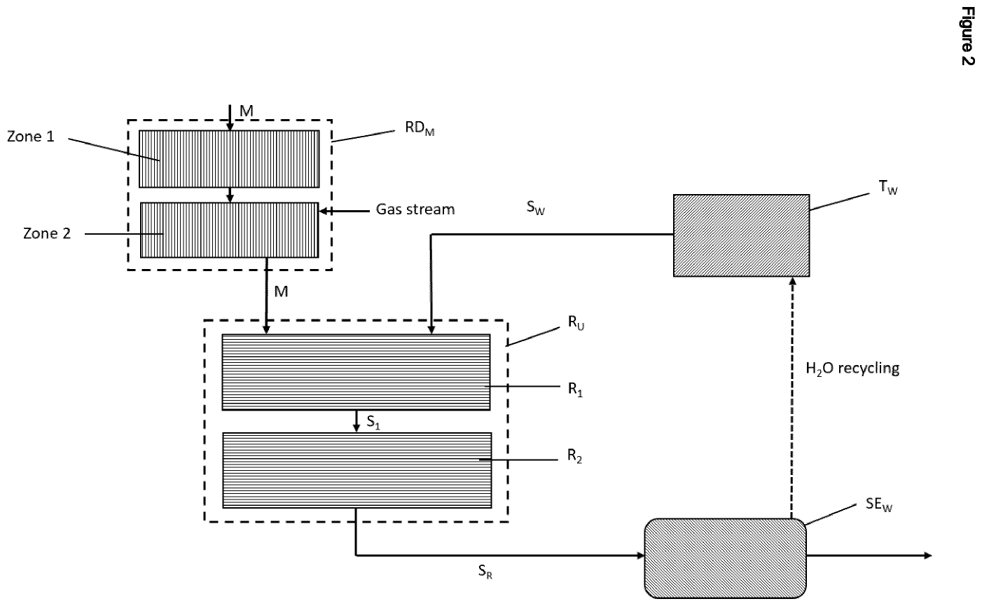

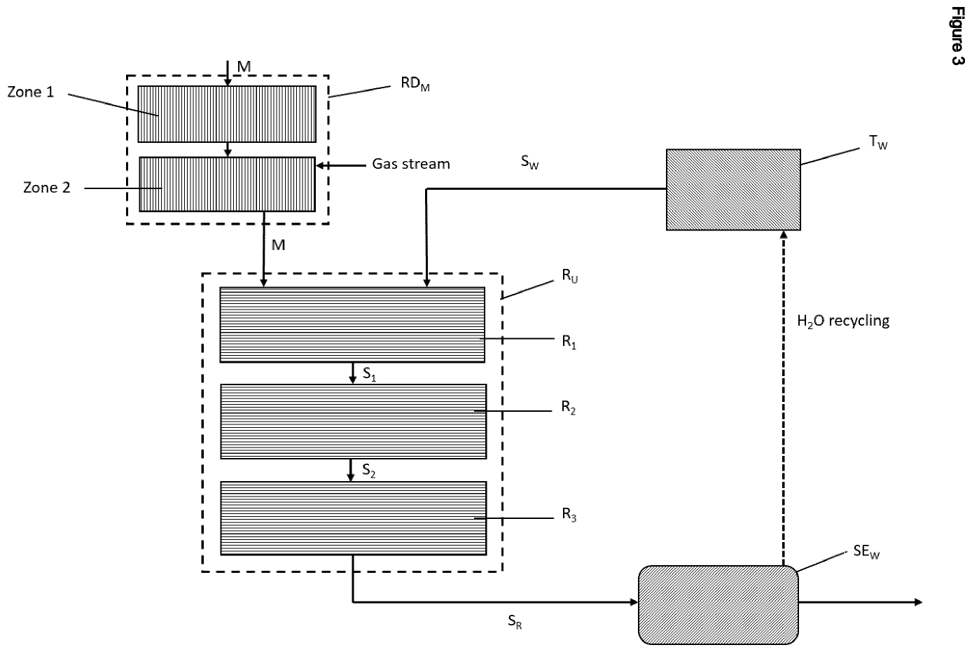

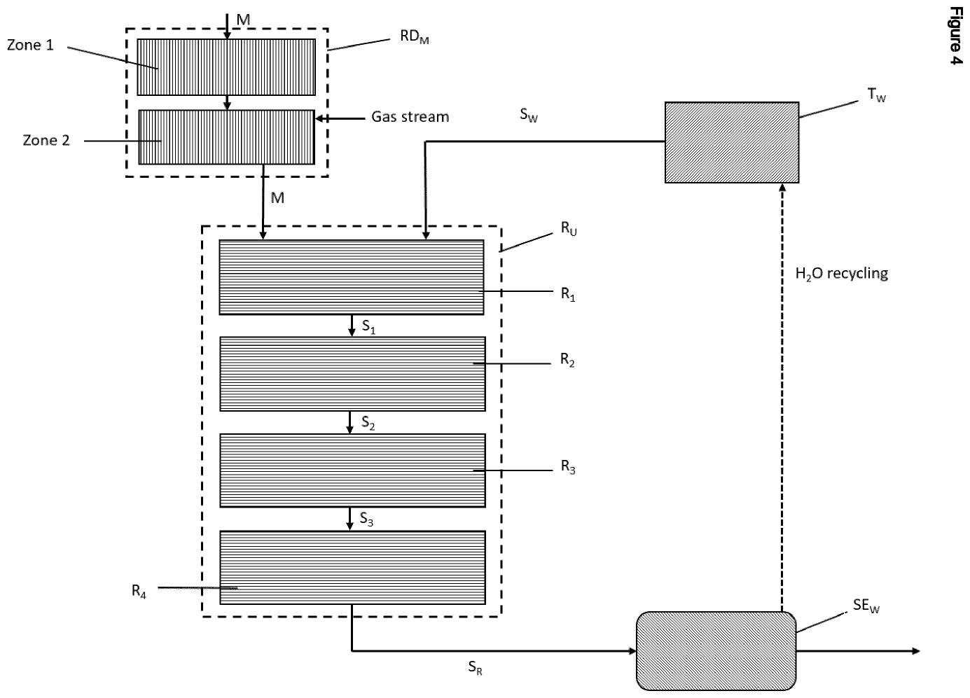

The apparatus comprises a reactor unit R_{U} comprising one chemical rector R₁, preferably a batch-type reactor, and a separation means SE_{W} downstream of the reactor unit. The solid material M is fed via an inlet means I_{M} (not shown in FIG. 1) into the reactor unit R_{U}, in particular at the top of the reactor R₁, as well as the liquid aqueous stream S_{W}. The product stream S_{R} containing ε-caprolactam dissolved in water is then removed from the bottom of reactor R₁ and is further passed through the separation means SE_{W} to separate S_{R} from water. Further, water removed from S_{R} is preferably recycled such that it can be introduced in S_{W} prior to entering the reaction unit R_{U} and in particular R₁. The water can be stored in the tank T_{W}. Furthermore, upstream of the reactor R₁ in the apparatus is a two-zone receiving and discharge means RD_{M} for metering the solid material M into the reactor unit R_{U} and in particular the reactor R₁, wherein zone 1 of RD_{M} is arranged vertically above zone 2 of RD_{M} and comprises inlet means I_{RD} (not shown in FIG. 1) for receiving the solid material M, wherein zone 2 comprises outlet means O_{RD} (not shown in FIG. 1) for removing the solid material from RD_{M}, the outlet means O_{RD} being connected via a connecting line with the inlet means I_{M} (not shown in FIG. 1) of the reactor R₁, wherein the RD_{M} further comprises closing and opening means arranged between zone 1 and zone 2 of the RD_{M}. Further a gas stream, preferably a high-pressure gas stream, is introduced in zone 2 of RD_{M}. The solid material M is transferred from zone 2 of RD_{M} to R₁ by gravity. Finally, the product stream S_{R} exiting the separation means SE_{W} can be further treated (not shown in FIG. 1).

FIG. 2 is a schematic representation of an apparatus used for the process according to the invention.

The apparatus comprises a reactor unit R_{U} comprising two chemical rectors R₁ and R₂, R₁ is arranged vertically above R₂, and a separation means SE_{W} downstream of the reactor unit. R₁ is a batch mode reactor and R₂ is a continuous mode reactor. The solid material M is fed via an inlet means I_{M} (not shown in FIG. 2) into R₁ as well as a liquid aqueous stream S_{W} comprising water. An aqueous liquid product stream S₁ is removed at the bottom of R₁ and fed at the top of R₂. The aqueous liquid product stream S₁ is transferred from R₁ to R₂ by gravity (no pumping, no conveyor means). The product stream S_{R} containing ε-caprolactam dissolved in water is then removed from the bottom of reactor R₂ and is further passed through the separation means SE_{W} to separate S_{R} from water. Further, water removed from S_{R} is preferably recycled such that it can be added to S_{W} prior to entering the reaction unit R_{U} and in particular R₁. The water can be stored in the tank T_{W}. Furthermore, upstream of the reactor unit R_{U} in the apparatus is a two-zone receiving and discharge means RD_{M} for metering the solid material M into the reactor unit R_{U} and in particular the reactor R₁, wherein zone 1 of RD_{M} is arranged vertically above zone 2 of RD_{M} and comprises inlet means I_{RD} (not shown in FIG. 2) for receiving the solid material M, wherein zone 2 comprises outlet means O_{RD} (not shown in FIG. 2) for removing the solid material M from RD_{M}, the outlet means O_{RD} being connected via a connecting line with the inlet means I_{M} (not shown in FIG. 2) of the reactor R₁, wherein the RD_{M} further comprises closing and opening means arranged between zone 1 and zone 2 of the RD_{M}. Further a gas stream, preferably a high-pressure gas stream, is introduced in zone 2 of RD_{M}. The solid material M is transferred from zone 2 of RD_{M} to R₁ by gravity. Finally, the product stream S_{R} exiting the separation means SE_{W} can be further treated (not shown in FIG. 2).

FIG. 3 is a schematic representation of an apparatus used for the process according to the invention.

The apparatus comprises a reactor unit R_{U} comprising three chemical rectors R₁, R₂ and R₃, R₁ is arranged vertically above R₂, R₂ is arranged vertically above R₃, and a separation means SE_{W} downstream of the reactor unit. R₁ and R₂ are batch mode reactors and R₃ is a continuous mode reactor. The solid material M is fed via an inlet means I_{M} (not shown in FIG. 3) into R₁ as well as a liquid aqueous stream S_{W} comprising water. An aqueous liquid product stream S₁ is removed at the bottom of R₁ and fed at the top of R₂. The aqueous liquid product stream S₁ is transferred from R₁ to R₂ by gravity (no pumping, no conveyor means). An aqueous liquid product stream S₂ is removed at the bottom of R₂ and fed at the top of R₃. The aqueous liquid product stream S₂ is transferred from R₂ to R₃ by gravity (no pumping, no conveyor means). The product stream S_{R} containing ε-caprolactam dissolved in water is then removed from the bottom of reactor R₃ and is further passed through the separation means SE_{W} to separate S_{R} from water. Further, water removed from S_{R} is preferably recycled such that it can be added to S_{W} prior to entering the reaction unit R_{U} and in particular R₁. The water can be stored in the tank T_{W}. Furthermore, upstream of the reactor unit R_{U} in the apparatus is a two-zone receiving and discharge means RD_{M} for metering the solid material M into the reactor unit R_{U} and in particular the reactor R₁, wherein zone 1 of RD_{M} is arranged vertically above zone 2 of RD_{M} and comprises inlet means I_{RD} (not shown in FIG. 3) for receiving the solid material M, wherein zone 2 comprises outlet means O_{RD} (not shown in FIG. 3) for removing the solid material M from RD_{M}, the outlet means O_{RD} being connected via a connecting line with the inlet means I_{M} (not shown in FIG. 3) of the reactor R₁, wherein the RD_{M} further comprises closing and opening means arranged between zone 1 and zone 2 of the RD_{M}. Further a gas stream, preferably a high-pressure gas stream, is introduced in zone 2 of RD_{M}. The solid material M is transferred from zone 2 of RD_{M} to R₁ by gravity. Finally, the product stream S_{R} exiting the separation means SE_{W} can be further treated (not shown in FIG. 3).

FIG. 4 is a schematic representation of an apparatus used for the process according to the invention.

The apparatus comprises a reactor unit R_{U} comprising three chemical rectors R₁, R₂, R₃ and R₄, R₁ is arranged vertically above R₂, R₂ is arranged vertically above R₃, R₃ is arranged vertically above R₄, and a separation means SE_{W} downstream of the reactor unit. R₁, R₂ and R₃ are batch mode reactors and R₄ is a continuous mode reactor. The solid material M is fed via an inlet means I_{M} (not shown in FIG. 4) into R₁ as well as a liquid aqueous stream S_{W} comprising water. An aqueous liquid product stream S₁ is removed at the bottom of R₁ and fed at the top of R₂. The aqueous liquid product stream S₁ is transferred from R₁ to R₂ by gravity (no pumping, no conveyor means). An aqueous liquid product stream S₂ is removed at the bottom of R₂ and fed at the top of R₃. The aqueous liquid product stream S₂ is transferred from R₂ to R₃ by gravity (no pumping, no conveyor means). An aqueous liquid product stream S₃ is removed at the bottom of R₃ and fed at the top of R₄. The aqueous liquid product stream S₃ is transferred from R₃ to R₄ by gravity (no pumping, no conveyor means). The product stream S_{R} containing ε-caprolactam dissolved in water is then removed from the bottom of reactor R₄ and is further passed through the separation means SE_{W} to separate S_{R} from water. Further, water removed from S_{R} is preferably recycled such that it can be added to S_{W} prior to entering the reaction unit R_{U} and in particular R₁. The water can be stored in the tank T_{W}. Furthermore, upstream of the reactor unit R_{U} in the apparatus is a two-zone receiving and discharge means RD_{M} for metering the solid material M into the reactor unit R_{U} and in particular the reactor R₁, wherein zone 1 of RD_{M} is arranged vertically above zone 2 of RD_{M} and comprises inlet means I_{RD} (not shown in FIG. 4) for receiving the solid material M, wherein zone 2 comprises outlet means O_{RD} (not shown in FIG. 4) for removing the solid material M from RD_{M}, the outlet means O_{RD} being connected via a connecting line with the inlet means I_{M} (not shown in FIG. 4) of the reactor R₁, wherein the RD_{M} further comprises closing and opening means arranged between zone 1 and zone 2 of the RD_{M}. Further a gas stream, preferably a high-pressure gas stream, is introduced in zone 2 of RD_{M}. The solid material M is transferred from zone 2 of RD_{M} to R₁ by gravity. Finally, the product stream S_{R} exiting the separation means SE_{W} can be further treated (not shown in FIG. 4).

1-15. (canceled)

16. A process for hydrolytically depolymerizing a polyamide prepared from ε-caprolactam, said polyamide being contained in a solid material M, the process comprising
(i) providing the solid material M containing the polyamide, M having a temperature T_{M}, wherein T_{M}<T_{P}, T_{P} being the melting point of the polyamide;
(ii) providing a liquid aqueous stream S_{W}, wherein from 90 to 100 weight-% of S_{W} consist of water and wherein S_{W} has a temperature T_{SW}, wherein T_{SW}>T_{P};
(iii) feeding the solid material M provided according to (i) and the liquid aqueous stream S_{W} provided according to (ii) into a chemical reactor unit R_{U}, obtaining a mixture;
(iv) subjecting the mixture obtained according to (iii) in R_{U} to a depolymerization temperature T_{D} at a depolymerization pressure p_{D}, wherein T_{M}<T_{D}<T_{SW}, obtaining an aqueous mixture containing ε-caprolactam dissolved in water;
(v) removing an aqueous liquid stream S_{R} from R_{U}, S_{R} containing ε-caprolactam dissolved in water.

17. The process of claim 16, wherein T_{D} is in the range of from 230 to 320° C.

18. The process of claim 16, wherein ΔT=T_{SW}−T_{D} and ΔT is in the range of from 10 to 70° C.

19. The process of claim 16, wherein M comprises waste material.

20. The process of claim 16, wherein according to (iii), M and S_{W} are fed into R_{U} at a mixing ratio m_{W}/kg:m_{P}/kg, defined as the amount of water contained in S₁, m_{W}, relative the mass of polyamide contained in M, m_{P}, in the range of from 1:1 to 20:1.

21. The process of claim 16, wherein prior to being fed into R_{U}, M is neither subjected to melting nor subjected to mashing.

22. The process of claim 16, wherein the chemical reactor unit R_{U} comprises z chemical reactors Rᵢ, i=1 . . . z, wherein z is in the range of from 1 to 10.

23. The process of claim 22, wherein z>1 and z reactors Rᵢ are serially coupled, wherein
according to (iii), M and S_{W} are fed into Rᵢ, i=1;
an aqueous liquid stream Sᵢ containing ε-caprolactam dissolved in water is removed from Rᵢ and fed into Rᵢ₊₁, i<z;
according to (v), S_{R} is removed from R_{z};
wherein in every reactor Rᵢ, a depolymerization temperature T_{Di} at a depolymerization pressure p_{Di} are maintained, wherein, independently of each other, T_{Di} is in the range of from 230 to 320° C.

24. The process of claim 22, wherein the reactors R₁ to R_{y-1} are operated in batch mode and the reactors R_{y} to R_{z} are operated in continuous mode, wherein y>1 and y≤z.

25. The process of claim 16, wherein providing a liquid aqueous stream S_{W} according to (ii) comprises
(ii.1) separating at least a part of the water contained in the aqueous liquid stream S_{R} removed from R_{U};
(ii.2) feeding at least a part of the water separated according to (ii.1) back into the process as part of the stream S_{W}.

26. The process of claim 16, wherein providing the solid material M containing the polyamide according to (i) comprises
(i.1) providing the solid material in a first zone of a two-zone receiving and discharge means RD_{M}, wherein the temperature of M in said first zone is in the range of from −10 to 50° C.;
(i.2) passing the solid material provided in the first zone to a second zone of the RD_{M}, wherein said second zone comprises outlet means being connected via a connecting line with the reactor unit R_{U};
(i.3) feeding a gas stream into the second zone of the RD_{M} comprising the solid material, said gas stream having a pressure p_{G} with p_{G}>p_{D};
(i.4) passing the solid material from the second zone of the RD_{M} into the connecting line according to (i.2).

27. An apparatus for carrying out a process for hydrolytically depolymerizing a polyamide prepared from ε-caprolactam, said polyamide being contained in a solid material M, the apparatus comprising
(a) a reactor unit R_{U} comprising z chemical reactors Rᵢ, i=1 . . . z, wherein z is in the range of from 2 to 8;
wherein the z reactors Rᵢ are serially coupled and vertically arranged, with R₁ being the top-most reactor and R_{z} being the bottom-most reactor;
wherein R₁ comprises inlet means I_{M} for feeding the solid material M into R₁ and inlet means I_{SW} for feeding a liquid aqueous stream S_{W} into R₁, said inlet means being located at the top of R₁;
wherein every reactor Rᵢ, comprises outlet means O_{Ri} for removing an aqueous liquid product stream Sᵢ from Rᵢ;
wherein a reactor Rᵢ, i>1, comprises inlet means I_{Ri} for feeding the aqueous liquid product stream Sᵢ₋₁ removed from Rᵢ₋₁ via a connecting line Cᵢ₋₁ into Rᵢ;
wherein the outlet stream removed from R_{z} is an aqueous liquid stream S_{R}, S_{R} containing ε-caprolactam dissolved in water.

28. The apparatus of claim 27, wherein at least 1 are configured as non-stirred reactors or as non-circulation reactors.

29. The apparatus of claim 27, further comprising
(b) separating means SE_{W} for separating water from S_{R};
wherein said separating means SE_{W} is arranged downstream of R_{U}, comprises inlet means I_{SE} for passing S_{R} into SE_{W}, the I_{SE} being connected via a connecting line with the outlet means O_{R} of the reactor R_{z}; and further comprise outlet means O_{SE} for removing water from SE_{W}, the O_{SE} being connected via a connecting line with the inlet means I_{SW} of the reactor R₁.

30. The apparatus of claim 27, further comprising
(c) a two-zone receiving and discharge means RD_{M} for metering the solid material M to the reactor unit R_{U};
wherein said two-zone receiving and discharge means RD_{M} is arranged upstream of R_{U},
wherein the first zone of RD_{M} is arranged vertically above the second zone of RD_{M} and comprises inlet means I_{RD} for receiving the solid material M, wherein the second zone of RD_{M} comprises outlet means O_{RD} for removing the solid material from RD_{M}, the outlet means O_{RD} being connected via a connecting line with the inlet means I_{M} of the reactor R₁, wherein the RD_{M} further comprises closing and opening means arranged between the first zone and the second zone of the RD_{M}.
